(19) United States
(12) Patent Application Publication
(10) Pub. No.: US 2020/0187565 A1
(43) Pub. Date: Jun. 18, 2020

(54) PERSONAL VAPORIZING DEVICE HAVING MULTIPLE METHODS FOR ACTIVATING THE DEVICE

(71) Applicant: Lunatech, LLC, Studio City, CA (US)

(72) Inventors: Josh Williams, Studio City, CA (US); David Christensen, Studio City, CA (US); Mike Elam, Thousand Oaks, CA (US); Alex Gilbert, Thousand Oaks, CA (US); Ravi Sawhney, Thousand Oaks, CA (US)

(21) Appl. No.: 16/217,879

(22) Filed: Dec. 12, 2018

(51) Int. Cl. A24F 47/00; A24F 7/00; A61M 15/00; G05B 19/4155; B05B 17/06

(52) U.S. Cl. CPC A24F 47/002; A24F 7/00; A61M 15/0021; G05B 2219/33206; G05B 19/4155; B05B 17/06; A61M 15/0085

(57) ABSTRACT

The present disclosure is directed to a personal vaporizing device having multiple methods for activating the personal vaporizing device. In one embodiment, the personal vaporizing device may be activated based on user preferences. In another embodiment, the personal vaporizing device may be activated based on an operating characteristic of the device and/or vaporizable material.

### BACKGROUND

The present disclosure is directed to a personal vaporizing device having multiple methods for activating the personal vaporizing device. In one embodiment, the personal vaporizing device may be activated based on user preferences. In another embodiment, the personal vaporizing device may be activated based on an operating characteristic of the device and/or vaporizable material.

In recent years, portable electronic vaporizers have gained popularity among users who vaporize herbal extracts to inhale the vapors emitted therefrom. Vaporization is an alternative to burning (smoking) that avoids the inhalation of may irritating toxic and carcinogenic by-products. With little or no smoke produced and generally cooler temperatures than what occurs when material is burned, less material is required to achieve a given level of effect. Hence the irritating and harmful effects of smoking are reduced, as is secondhand smoke.

Many of the battery-powered vaporizers include a reusable battery-containing device portion that connects to one or more cartridges or containers containing the consumable vaporizable material or liquid solution (“E-liquid”). The main ingredients of e-liquids are usually a mixture of propylene glycol, vegetable glycerin, and/or polyethylene glycol, often with differing levels of alcohol mixed with concentrated or extracted flavorings, and a variable concentration of nicotine. In some embodiments, the e-liquid may contain medicinal agents, recreational agents, nutraceuticals, wellness agents, and the like. There is variability in the purity, kinds, and concentrations of ingredients used in the liquids, and as well as the shape, size, and other specifications of the e-liquid container.

Typically, a personal vaporizing device is designed and/or authorized to work with only certain e-liquid containers. If a user attempts to use an e-liquid container not designed for use with a specific personal vaporizing device, it could result in damage to the vaporizing device and/or injury to the user. In addition, certain e-liquid compositions, such as those containing prescription medications or recreational additives, should only be consumed by authorized user. As an example, an e-liquid composition containing a specified prescription should only be used by the person to whom such medication was prescribed. In addition, an e-liquid composition containing nicotine or other recreational additives should be limited in use to adults, such that minors would be prevented from using such compositions.

Therefore, it would be desirable to provide a personal vaporizing device that has multiple methods of activation dependent upon user preference, a characteristic of a user of the device, and/or an operating characteristic of the device and/or vaporizable material to prevent unauthorized or misuse thereof.

### SUMMARY

The following presents a simplified overview of the example embodiments in order to provide a basic understanding of some embodiments of the example embodiments. This overview is not an extensive overview of the example embodiments. It is intended to neither identify key or critical elements of the example embodiments nor delineate the scope of the appended claims. Its sole purpose is to present some concepts of the example embodiments in a simplified form as a prelude to the more detailed description that is presented hereinbelow. It is to be understood that both the following general description and the following detailed description are exemplary and explanatory only and are not restrictive.

In accordance with the embodiments disclosed herein, the present disclosure is directed to a personal vaporizing device that has multiple methods of activation dependent upon user preference, a characteristic of a user of the device, and/or an operating characteristic of the device and/or vaporizable material.

In an embodiment, there is provided a personal vaporizer comprising a device processor operable to control the personal vaporizer, wherein the device processor is operable to generate an activation command to initiate a vaporization process, and a container configured to store a vaporizable liquid composition. The personal vaporizer further comprises an ultrasonic vaporizing component operatively coupled to the device processor and controlled in part by the device processor, wherein the ultrasonic vaporizing component is in fluid communication with the container for receiving a selected amount of vaporizable liquid composition from the container, wherein the ultrasonic vaporizing component comprises an ultrasonic vibration element operable to produce ultrasonic vibrations to vaporize at least a portion of the vaporizable liquid composition received therein. The personal vaporizer also comprises a mouthpiece coupled to the ultrasonic vaporizing component and configured to receive vapor generated by the ultrasonic vaporizing component, the mouthpiece operable to expel the generated vapor from the ultrasonic vaporizing component, an input/output device operatively coupled to the device processor; wherein the input/output device is configured to receive a plurality of data for transmission to the device processor, wherein the input/output device is configured to transmit a plurality of data generated by the device processor, and a power source operatively coupled to the ultrasonic vaporizing component, wherein the power source is operable to generate an electric current for operation of the ultrasonic vaporizing component. The device processor is operable to receive a plurality of device activation parameters for controlling activation of the vaporization process and generate at least one device activation control signal in accordance with at least a portion of the plurality of device activation parameters. The device processor is further operable to transmit the at least one device activation control signal to the ultrasonic vaporizing component to initiate the vaporization process in accordance the at least one device activation control signal.

In an embodiment, the input/output device comprises a user interface, wherein the device processor is operable to receive at least a portion of the plurality of device activation parameters from an associated user via the user interface. In another embodiment, the input/output device is configured to receive at least a portion of the plurality of device activation parameters for controlling activation of a vaporization process from a remote device.

In one embodiment, the plurality of device activation parameters is selected from the group consisting of type of activation mode, a location of the personal vaporizer, a time of day, a type of vaporizable liquid composition stored in the container, an operational parameter of the container, desired vapor output, an operational parameter of the mouthpiece, and combinations thereof.

In various implementations, the device processor is further operable to detect a plurality of status data associated with at least one operational characteristic of at least one of the container, the mouthpiece, the ultrasonic vaporizing component and combinations thereof. The device processor is also operable to determine, based on the at least a portion of the detected status data, at least one operational parameter of the personal vaporizer, determine, based on the at least one operational parameter, at least one device activation parameter for controlling activation of the vaporization process, and generate at least one device activation control signal in accordance with the at least one device activation parameter.

In one embodiment, the personal vaporizer further comprises a sensing component operatively connected to the device processor and controlled in part by the device processor, wherein the sensing component is operable to detect a plurality of status data associated with at least one operational characteristic of the personal vaporizer and transmit at least a portion of the detected status data to the device processor. In a preferred embodiment, the device processor is further operable to receive at least a portion of the detected status data. The device processor is also operable to determine, based on the received status data, at least one operational parameter of the personal vaporizer, determine, based on the at least one operational parameter, at least one device activation parameter for controlling activation of the vaporization process, and generate at least one device activation control signal in accordance with the at least one device activation parameter.

In yet another embodiment, the sensing component is operable to detect a plurality of status data associated at least one operational characteristic of the mouthpiece and transmit at least a portion of the detected mouthpiece status data to the device processor. In such embodiment, the device processor is further operable to determine, based on the received mouthpiece status data, at least one operational parameter of the mouthpiece, determine, based on the at least one operational parameter, at least one device activation parameter for controlling activation of the vaporization process, and generate at least one device activation control signal in accordance with the at least one device activation parameter.

In another embodiment, the sensing component is operable to detect a plurality of status data associated at least one operational characteristic of the container and transmit at least a portion of the detected container status data to the device processor. In such embodiment, the device processor is further operable to determine, based on the received container status data, at least one operational parameter of the container, determine, based on the at least one operational parameter, at least one device activation parameter for controlling activation of the vaporization process, and generate at least one device activation control signal in accordance with the at least one device activation parameter.

In one embodiment, the sensing component is selected from the group of sensing components consisting of: a biochemical/chemical sensor, a thermal sensor, a radiation sensor, a mechanical sensor, an optical sensor, a magnetic sensor, an electrical sensor, and combinations thereof.

In accordance with the embodiments disclosed herein, there is provided a method for activating a personal vaporizer to initiate a vaporization process. The personal vaporizer comprises (a) a device processor operable for controlling the personal vaporizer; (b) a container configured to store a vaporizable liquid composition; (c) an ultrasonic vaporizing component comprising an ultrasonic vibration element operable to produce ultrasonic vibrations to vaporize at least a portion of the vaporizable liquid composition received therein; (d) a mouthpiece configured to receive vapor generated by the ultrasonic vaporizing component and expel the generated vapor from the ultrasonic vaporizing component; (e) an input/output device configured to receive a plurality of data for transmission to the device processor and to transmit a plurality of data generated by the device processor; and (f) a power source operable to generate a variable strength electrical current for operation of the ultrasonic vaporizing component.

The method comprises receiving, at the device processor a plurality of device activation parameters for controlling activation of the vaporization process, and generating, via the device processor at least one device activation control signal in accordance with at least a portion of the plurality of device activation parameters. The at least one device activation control signal is transmitted via the input/output device to the ultrasonic vaporizing component to initiate the vaporization process in accordance the at least one device activation control signal. A vaporization process is initiated by the ultrasonic vaporizing component to vaporize at least a portion of the vaporizable liquid composition.

In one embodiment, the input/output device comprises a user interface, and the method further comprises receiving at least a portion of the plurality of device activation parameters from an associated user via the user interface.

In one embodiment, the method comprises receiving, via the input/output device, at least a portion of the plurality of device activation parameters for controlling activation of a vaporization process from a remote device.

In another embodiment, the plurality of device activation parameters is selected from the group consisting of type of activation mode, a location of the personal vaporizer, a time of day, a type of vaporizable liquid composition stored in the container, an operational parameter of the container, desired vapor output, an operational parameter of the mouthpiece, and combinations thereof.

In yet another embodiment, the method further comprises detecting, via the device processor, a plurality of status data associated with at least one operational characteristic of at least one of the container, the mouthpiece, the ultrasonic vaporizing component and combinations thereof. The device processor, based on the at least a portion of the detected status data, determines at least one operational parameter of the personal vaporizer. The device processor, based on the at least one operational parameter, determines at least one device activation parameter for controlling activation of the vaporization process, and generates at least one device activation control signal in accordance with the at least one device activation parameter.

In a further embodiment, the personal vaporizer further comprises a sensing component. In such embodiment, the method further comprises detecting, via the sensing component, a plurality of status data associated with at least one operational characteristic of the personal vaporizer and transmitting at least a portion of the detected status data to the device processor.

In one embodiment, the method comprises receiving, at the device processor, at least a portion of the detected status data from the sensing component. The device processor, based on the at least a portion of the received status data, determines at least one operational parameter of the personal vaporizer. The device processor, based on the at least one operational parameter, determines at least one device activation parameter for controlling activation of the vaporization process, and generates at least one device activation control signal in accordance with the at least one device activation parameter.

In a preferred embodiment, the method comprises detecting, by the sensing component, a plurality of status data associated at least one operational characteristic of the mouthpiece and transmitting at least a portion of the detected mouthpiece status data to the device processor. The device processor, based on the at least a portion of the received mouthpiece status data, determines at least one operational parameter of the personal vaporizer. The device processor, based on the at least one operational parameter, determines at least one device activation parameter for controlling activation of the vaporization process, and generates at least one device activation control signal in accordance with the at least one device activation parameter.

In another preferred embodiment, the method comprises detecting, by the sensing component, a plurality of status data associated at least one operational characteristic of the container and transmitting at least a portion of the detected container status data to the device processor. The device processor, based on the at least a portion of the received container status data, determines at least one operational parameter of the personal vaporizer. The device processor, based on the at least one operational parameter, determines at least one device activation parameter for controlling activation of the vaporization process, and generates at least one device activation control signal in accordance with the at least one device activation parameter.

In an embodiment, there is provided a personal vaporizer comprising a device processor operable to control the personal vaporizer, wherein the device processor is operable to generate an activation command to initiate a vaporization process, and a container configured to store a vaporizable liquid composition. The personal vaporizer further comprises an ultrasonic vaporizing component operatively coupled to the device processor and controlled in part by the device processor, wherein the ultrasonic vaporizing component is in fluid communication with the container for receiving a selected amount of vaporizable liquid composition from the container, wherein the ultrasonic vaporizing component comprises an ultrasonic vibration element operable to produce ultrasonic vibrations to vaporize at least a portion of the vaporizable liquid composition received therein. The personal vaporizer also comprises a mouthpiece coupled to the ultrasonic vaporizing component and configured to receive vapor generated by the ultrasonic vaporizing component, the mouthpiece operable to expel the generated vapor from the ultrasonic vaporizing component. The personal vaporizer further comprises a sensing component operatively connected to the device processor and controlled in part by the device processor, wherein the sensing component is operable to detect a plurality of status data associated with at least one operational characteristic of at least one of the mouthpiece, the container, and the ultrasonic vaporizing component and combinations thereof and transmit at least a portion of the detected status data to the device processor. The personal vaporizer also includes a power source operatively coupled to the ultrasonic vaporizing component, wherein the power source is operable to generate an electric current for operation of the ultrasonic vaporizing component. The device processor is operable to determine, based on the received status data, at least one operational parameter of the personal vaporizer, determine, based on the at least one operational parameter, at least one device activation parameter for controlling activation of the vaporization process, and generate at least one device activation control signal in accordance with the at least one device activation parameter.

The device processor is further operable to transmit the at least one device activation control signal to the ultrasonic vaporizing component to initiate the vaporization process in accordance the at least one device activation control signal.

Still other advantages, embodiments, and features of the subject disclosure will become readily apparent to those of ordinary skill in the art from the following description wherein there is shown and described a preferred embodiment of the present disclosure, simply by way of illustration of one of the best modes best suited to carry out the subject disclosure. As it will be realized, the present disclosure is capable of other different embodiments and its several details are capable of modifications in various obvious embodiments all without departing from, or limiting, the scope herein. Accordingly, the drawings and descriptions will be regarded as illustrative in nature and not as restrictive.

### BRIEF DESCRIPTION OF THE DRAWINGS

The drawings are of illustrative embodiments. They do not illustrate all embodiments. Other embodiments may be used in addition or instead. Details which may be apparent or unnecessary may be omitted to save space or for more effective illustration. Some embodiments may be practiced with additional components or steps and/or without all of the components or steps which are illustrated. When the same numeral appears in different drawings, it refers to the same or like components or steps.

FIG. 1 illustrates block diagrams of one embodiment of personal vaporizing device according to the present disclosure.

FIG. 2 is an illustration of another embodiment of a personal vaporizing device according to the present disclosure.

FIG. 3 is a flow chart illustrating one method for activating a personal vaporizing device according to the present disclosure.

FIG. 4 is a flow chart illustrating another method for activating a personal vaporizing device according to the present disclosure.

FIG. 5 is a flow chart illustrating another method for activating a personal vaporizing device according to the present disclosure.

### DETAILED DESCRIPTION OF THE ILLUSTRATIVE EMBODIMENTS

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

As used herein, the term “by weight,” when used in conjunction with a component, unless specially stated to the contrary is based on the total weight of the formulation or composition in which the component is included. For example, if a particular element or component in a composition or article is said to have 8% by weight, it is understood that this percentage is in relation to a total compositional percentage of 100%.

A weight percent of a component, or weight %, or weight percent, or weight % (percent) unless specifically stated to the contrary, is based on the total weight of the formulation or composition in which the component is included.

References in the specification and concluding claims to parts by weight of a particular element or component in a composition or article, denotes the weight relationship between the element or component and any other elements or components in the composition or article for which a part by weight is expressed. Thus, in a composition or a selected portion of a composition containing 2 parts by weight of component X and 5 parts by weight component Y, X and Y are present at a weight ratio of 2:5, and are present in such ratio regardless of whether additional components are contained in the composition.

In the following description, certain terminology is used to describe certain features of one or more embodiments. For purposes of the specification, unless otherwise specified, the term “substantially” refers to the complete or nearly complete extent or degree of an action, characteristic, property, state, structure, item, or result. For example, in one embodiment, an object that is “substantially” located within a housing would mean that the object is either completely within a housing or nearly completely within a housing. The exact allowable degree of deviation from absolute completeness may in some cases depend on the specific context. However, generally speaking, the nearness of completion will be so as to have the same overall result as if absolute and total completion were obtained. The use of “substantially” is also equally applicable when used in a negative connotation to refer to the complete or near complete lack of an action, characteristic, property, state, structure, item, or result.

As used herein, the term “substantially,” in, for example, the context “substantially free” refers to a composition having less than about 10% by weight, e.g., less than about 5%, less than about 1%, less than about 0.5% by weight, less than about 0.1% by weight, less than about 0.05% by weight, or less than about 0.01% by weight of the stated material, based on the total weight of the composition.

It is further understood that the term “substantially,” when used in reference to a composition, refers to at least about 60% by weight, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by weight, based on the total weight of the composition, of a specified feature, component, or a combination of the components. It is further understood that if the composition comprises more than one component, the two or more components may be present in any ratio predetermined by one of ordinary skill in the art. For example, and without limitation, the composition comprising substantially water and natural flavor, unless specifically recited, may comprise water and natural flavor in any ratio predetermined by one of ordinary skill in the art.

As used herein, the terms “approximately” and “about” generally refer to a deviance of within 5% of the indicated number or range of numbers. In one embodiment, the term “approximately” and “about”, may refer to a deviance of between 0.001-10% from the indicated number or range of numbers.

As used herein, the terms “electronic liquid,” “natural-based liquid composition,” or “e-liquid” may be used interchangeably and refer to a mixture used in a vapor product, such as an personal vaporizing device. In some embodiments, the personal vaporizing device may include without limitation electronic cigarettes, electronic pipes, electronic cigars, and the like.

Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all embodiments of this application including, but not limited to, steps in disclosed methods. Thus, if there are a variety of additional steps that may be performed it is understood that each of these additional steps may be performed with any specific embodiment or combination of embodiments of the disclosed methods.

The present methods and systems may be understood more readily by reference to the following detailed description of preferred embodiments and the examples included therein and to the Figures and their previous and following description.

As will be appreciated by one skilled in the art, the methods and systems may take the form of an entirely hardware embodiment, an entirely software embodiment, or an embodiment combining software and hardware embodiments. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, may be implemented by computer program instructions. These computer program instructions may be loaded onto a general-purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that may direct a computer or other programmable data processing apparatus to function in a particular manner, such that the instructions stored in the computer-readable memory produce an article of manufacture including computer- readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, may be implemented by special purpose hardware-based computer systems that perform the specified functions or steps, or combinations of special purpose hardware and computer instructions.

Various embodiments are now described with reference to the drawings. In the following description, for purposes of explanation, numerous specific details are set forth in order to provide a thorough understanding of one or more embodiments. It may be evident, however, that the various embodiments may be practiced without these specific details. In other instances, well-known structures and devices are shown in block diagram form to facilitate describing these embodiments.

In various implementations,

In one embodiment,

In one embodiment,

In one embodiment, disclosed is personal vaporizing device (e.g., e-cigarette) enabled with a broad range of functionality options and may be configured to be activated based upon user preference and/or an operating characteristic of the device and/or vaporizable material. These functionalities are enabled by a microprocessor controller utilized to execute commands for system functionality, along with a memory, transmitter, software, storage, and power system. The personal vaporizing device itself may be outfitted with a heating element, cooling element, and a variety of attendant functionality options. Such options include networking and communication services, device monitoring, mixing, heating, cooling, refilling, aromatic, and other distribution functions, external monitoring, testing, powering options, portability, device effects including sound, imaging, light and graphical effects, remote and third-party control, symbiotic characteristics with other devices, and synchronicity among devices.

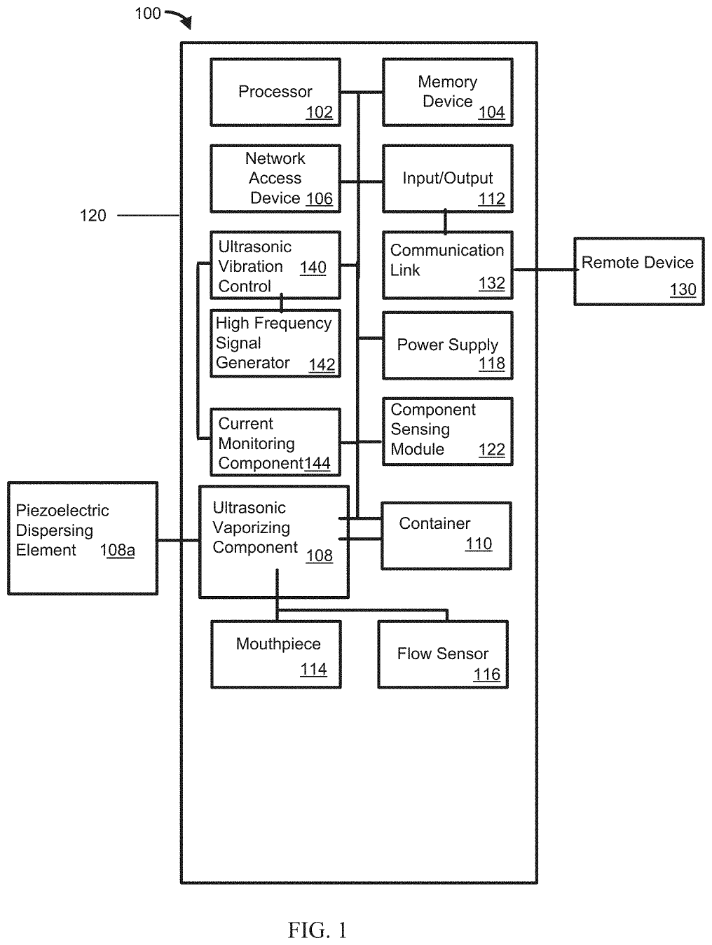

FIG. 1 is a block diagram of one embodiment of a personal vaporizing device 100 as described herein. The personal vaporizing device 100 may be, for example, an electronic cigarette, an electronic cigar, an electronic vapor device, a modified vapor device (also known as a mod), a micro-sized electronic vapor device, and the like. The personal vaporizing device 100 may comprise any suitable housing 120 for enclosing and protecting the various components disclosed herein. The personal vaporizing device 100 may comprise a processor 102 operable to control the operation of the personal vaporizing device 100. The processor 102 may be, or may comprise, any suitable microprocessor or microcontroller, for example, a low-power application-specific controller (ASIC) and/or a field programmable gate array (FPGA) designed or programmed specifically for the task of controlling a device as described herein, or a general purpose central processing unit (CPU), for example, one based on 80×86 architecture as designed by Intel™ or AMD™, or a system-on-a-chip as designed by ARM™. The processor 102 may be coupled (e.g., communicatively, operatively, etc.) to auxiliary devices or modules of the personal vaporizing device 100 using a bus or other coupling. The personal vaporizing device 100 may comprise power supply 118. The power supply 118 may comprise one or more batteries and/or other power storage device (e.g., capacitor) and/or a port for connecting to an external power supply. The one or more batteries may be rechargeable. The one or more batteries may comprise a lithium-ion battery (including thin film lithium ion batteries), a lithium-ion polymer battery, a nickel-cadmium battery, a nickel metal hydride battery, a lead-acid battery, ultra-capacitor, super-capacitor combinations thereof, and the like. For example, an external power supply may supply power to the personal vaporizing device 100 and a battery may store at least a portion of the supplied power.

The personal vaporizing device 100 may comprise a memory device 104 coupled to the processor 102. The memory device 104 may comprise a random-access memory (RAM) configured for storing program instructions and data for execution or processing by the processor 102 during control of the personal vaporizing device 100. When the personal vaporizing device 100 is powered off or in an inactive state, program instructions and data may be stored in a long-term memory, for example, a non-volatile magnetic optical, or electronic memory storage device (not shown). At least one of the RAM or the long-term memory may comprise a non-transitory computer-readable medium storing program instructions that, when executed by the processor 102, cause the personal vaporizing device 100 to perform all or part of one or more methods and/or operations described herein. Program instructions may be written in any suitable high-level language, for example, C, C++, C# or the Java™, and compiled to produce machine-language code for execution by the processor 102.

In one embodiment, the personal vaporizing device 100 may comprise a network access device 106 allowing the personal vaporizing device 100 to be coupled to one or more ancillary devices (not shown) such as via an access point (not shown) of a wireless telephone network, local area network, or other coupling to a wide area network, for example, the Internet. In that regard, the processor 102 may be configured to share data with the one or more ancillary devices via the network access device 106. The shared data may comprise, for example, usage data and/or operational data of the personal vaporizing device 100, a status of the personal vaporizing device 100, a status and/or operating condition of one or more the components of the personal vaporizing device 100, text to be used in a message, a product order, payment information, and/or any other data. Similarly, the processor 102 may be configured to receive control instructions from the one or more ancillary devices via the network access device 106. For example, a configuration of the personal vaporizing device 100, an operation of the personal vaporizing device 100, and/or other settings of the personal vaporizing device 100, may be controlled by the one or more ancillary devices via the network access device 106. For example, an ancillary device may comprise a server that may provide various services and another ancillary device may comprise a smartphone for controlling operation of the personal vaporizing device 100. In some embodiments, the smartphone or another ancillary device may be used as a primary input/output of the personal vaporizing device 100 such that data may be received by the personal vaporizing device 100 from the server, transmitted to the smartphone, and output on a display of the smartphone.

In an embodiment, the personal vaporizing device 100 may also comprise an input/output device 112 coupled to one or more of the processor 102, the network access device 106, the vaporizing component 108, and/or any other electronic component of the personal vaporizing device 100. Input may be received from a user or another device and/or output may be provided to a user or another device via the input/output device 112. The input/output device 112 may comprise any combinations of input and/or output devices such as buttons, knobs, keyboards, touchscreens, displays, light-emitting elements, a speaker, and/or the like. In an embodiment, the input/output device 112 may comprise an interface port (not shown) such as a wired interface, for example a serial port, a Universal Serial Bus (USB) port, an Ethernet port, or other suitable wired connection. The input/output device 112 may comprise a wireless interface (not shown), for example a transceiver using any suitable wireless protocol, for example Wi-Fi (IEEE 802.11), Bluetooth®, infrared, or other wireless standard. For example, the input/output device 112 may communicate with a smartphone via Bluetooth® such that the inputs and outputs of the smartphone may be used by the user to interface with the personal vaporizing device 100. In an embodiment, the input/output device 112 may comprise a user interface.

In an embodiment, the input/output device 112 may comprise a touchscreen interface and/or a biometric interface. For example, the input/output device 112 may include controls that allow the user to interact with and input information and commands to the personal vaporizing device 100. For example, with respect to the embodiments described herein, the input/output device 112 may comprise a touch screen display. User inputs to the touch screen display are processed by, for example, the input/output device 112 and/or the processor 102. The input/output device 112 may also be configured to process new content and communications to the personal vaporizing device 100. The touch screen display may provide controls and menu selections, and process commands and requests. The input/output device 112 and/or the processor 102 may receive and interpret commands and other inputs, interface with the other components of the personal vaporizing device 100 as required. In an embodiment, the touch screen display may enable a user to lock, unlock, or partially unlock or lock, the personal vaporizing device 100. The input/output device 112 may thus display information to a user such as a puff count, an amount of vaporizable material remaining in the container 110, battery remaining, signal strength, combinations thereof, and the like.

As shown in FIG. 1, in an embodiment, the personal vaporizing device 100 comprises a vaporizing component 108. The vaporizing component 108 is coupled to the vaporizable liquid container 110. The container 110 is configured to hold one or more vaporizable liquid compositions. The vaporizing component 108 may receive at least portion of the vaporizable liquid composition from the container 110 for vaporizing at least a portion of the liquid composition. In one embodiment, the vaporizing component 108 may nebulize or otherwise cause the vaporizable liquid composition from pre-filled container 110 to reduce in size into particulates.

In another embodiment, the vaporizing component 108 may comprise a piezoelectric dispersing element 108a. In some embodiments, the piezoelectric dispersing element 108a may be charged by a battery, and may be driven by a processor on a circuit board. The circuit board may be produced using a polyimide such as Kapton®, or other suitable material. The piezoelectric dispersing element 108a may comprise a thin metal disc which causes dispersion of the fluid fed into the dispersing element through vibration. Once in contact with the piezoelectric dispersing element 108a, the vaporizable liquid composition may be vaporized (e.g., turned into vapor or mist) and the vapor may be dispersed via a system pump and/or a sucking action of the user. In some embodiments, the piezoelectric dispersing element 108a may cause dispersion of the vaporizable liquid composition by producing ultrasonic vibrations. An electric field applied to a piezoelectric material within the piezoelectric dispersing element 108a may cause ultrasonic expansion and contraction of the piezoelectric material, resulting in ultrasonic vibrations to the disc. The ultrasonic vibrations may cause the vaporizable liquid composition to disperse, thus forming a vapor or mist from the vaporizable liquid composition.

In some embodiments, the connection between the power supply 118 and the piezoelectric dispersing element 108a may be facilitated using one or more conductive coils. The conductive coils may provide an ultrasonic power input to the piezoelectric dispersing element 108a. In some embodiments, the piezoelectric dispersing element 108a may comprise a piezoelectric material that may receive the ultrasonic signal transmitted from the power supply through the coils, and may cause cold vaporization (or atomization) of the vaporizable liquid by producing ultrasonic vibrations. An ultrasonic electric field applied to a piezoelectric material within the piezoelectric dispersing element 108a causes ultrasonic expansion and contraction of the piezoelectric material, resulting in ultrasonic vibrations according to the frequency of the signal. The vaporizable liquid composition may be vibrated by the ultrasonic energy produced by the piezoelectric dispersing element 108a, thus causing dispersal and/or atomization of the liquid.

In an embodiment, input from the input/output device 112 may be used by the processor 102 to cause the vaporizing component 108 to vaporize the vaporizable liquid composition. For example, a user may depress a button, causing the vaporizing component 108 to start vaporizing vaporizable liquid composition. A user may then draw on mouthpiece 114 to inhale the vapor. In various embodiments, the processor 102 may control vapor production and flow to the mouthpiece 114 based on data detected by a flow sensor 116. For example, as a user draws on the mouthpiece 114, the flow sensor 116 may detect the resultant pressure and provide a signal to the processor 102. In response, the processor 102 may cause the vaporizing component 108 to begin vaporizing the vaporizable liquid composition, terminate vaporizing the vaporizable liquid composition, and/or otherwise adjust a rate of vaporization of the vaporizable liquid composition.

The vaporizable liquid composition is comprised of any material or combination of materials that may be transformed into a vapor. For example, the vaporizable liquid composition may include water, solvents, active ingredients, ethanol, plant extracts, natural or artificial flavors, and/or vapor formers such as glycerin and propylene glycol.

In some embodiments, the vaporizable liquid composition may include one or more of propylene glycol, glycerin, and combinations. In other embodiments, the vaporizable liquid composition may be comprised of substantially all-natural ingredients. In yet other embodiment, vaporizable liquid composition may be comprised of primarily water. In another embodiment, the vaporizable liquid composition is substantially free of at least one of propylene glycol, vegetable glycerin, and combinations thereof.

In certain embodiments, the vaporizable liquid composition may comprise at least one supplementary component comprising a medicinal agent or element, a wellness agent or element, a recreational use agent or element, a flavoring agent, and any combinations of thereof.

In yet other embodiments, the wellness element may comprise a homeopathic remedy, a vitamin supplement, a nutraceutical, or any combination thereof. In certain embodiments, the medicinal element may comprise a pharmaceutical composition, a medication, a medicinal element, and the like. In some embodiments, the recreational element may comprise caffeine, nicotine, cannabis-based elements or compounds such as cannabinoids, taurine, salvia, kratum, kava, or any combination thereof.

In one embodiment, the vaporizable liquid composition may comprise at least one flavoring agent comprising a flavor of fruits, berries, spices, herbs, savory flavors, spicy flavors, sweet flavors, plant based flavors, and combinations thereof. In certain embodiments, the flavorings may comprise at least one of citrus flavorings, fruit flavorings, berry, spice flavorings, flower flavorings, herbaceous flavorings, vegetable flavorings, savory flavorings, sour flavorings, spicy flavorings, bitter flavorings, and combinations thereof.

As shown in FIG. 1, the personal vaporizing device 100 comprises a vaporizing component 108 that is in fluid communication with container 110 for receiving vaporizable liquid material therefrom. In one embodiment, the vaporizing component 108 includes a piezoelectric dispersing element 108a to vaporize at least a portion of the vaporizable liquid material received from the container 110. The piezoelectric dispersing element 108a may be operable to produce ultrasonic vibrations to vaporize at least a portion of the vaporizable liquid material received into the vaporizer component 108. While reference is made to personal vaporizing device 100, it is to be understood that the personal vaporizing may be any personal vaporizer in accordance with the present disclosure.

The processor 102 may be operable to generate at least one vaporizing control signal for controlling at least one operational parameter of the vaporizing component 108 for vaporizing the liquid composition received from container 110. In one embodiment, the at least one vaporizing control signal may be based on a type of material contained in container 110. In another embodiment, the at least one vaporizing control signal may be based on at least one operational characteristic of the personal vaporizing device 100. In yet another embodiment, the at least one vaporizing control signal may be based on a characteristic of a container 110, mouthpiece 114, and/or other component used in the operation of personal vaporizing device 100. In another embodiment, the at least one vaporizing control signal may be based on at least one characteristic of a user of the personal vaporizing device 100.

In one embodiment, the input/output device 112 may be configured to receive a plurality of remote control signals generated by a remote device 130 for controlling at least one operational parameter of the personal vaporizing device 100 and to transmit the plurality of received remote control signals to the processor 102 for controlling the operation of the personal vaporizing device 100 in response thereto. In one embodiment, the remote device 130 may generate at least one remote control signal for activating the personal vaporizing device 100. The at least one remote control signal may then be transmitted to the input/output device 112 via communication link 132. The at least one remote control signal is then transmitted to the processor 102, wherein the processor may generate least one control signal for activating the personal vaporizing device.

In operation, the personal vaporizing device 100 may obtain a plurality of vaporizing control parameters for controlling at least one operational parameter of the vaporizing component 108. The processor 102 may generate at least one vaporizing control signal in accordance with at least a portion of the plurality of vaporizing control parameters. The vaporizing component 108 may then withdraw a selected amount of vaporizable liquid material from the container 110. At least a portion of the withdrawn vaporizable liquid material may be vaporized by the vaporizing component 108 in accordance with the at least one vaporizing control signal.

As an example, the vaporizing control parameters include, but are not limited to, a type of vaporizable material stored in the container (water-based composition, contains propylene glycol/vegetable glycerin), a selected amount of vaporizable material withdrawn from container, desired vapor output (mixture, temperature, amount of vapor, etc.), power required to operate the vaporizing component, operational status of the personal vaporizing device 100; operational status of the vaporizing component 108, a location of the personal vaporizing device 100, at least one characteristic of a user of the personal vaporizing device 100, an activation mode of the personal vaporizing device 100, and combinations thereof.

Data relating to the plurality of vaporizing control parameters may be obtained by any suitable means. In a preferred embodiment, the processor 102 receives at least a portion of the vaporizing control parameters from an associated user, other computer system, device, network, or the like via the input/output device 112, through the network access device 106, sensor 116, via a computer readable medium, or combinations thereof.

In one embodiment, a user may input desired vaporizing control parameters via a user interface associated with the input/output device 112. The input/output device 112 may include the functionality to allow an associated user to select parameters, features or other options for the vaporizing control parameters.

In another embodiment, at least a portion of the plurality of vaporizing control parameters may be provided via a user interface associated with the remote device 130 and then transmitted to the input/output device 112 via communication link 132. As an example, a third party, such as a parent or guardian, health care professional, authorized retailer, authorized regulatory or governmental authority, and the like, may provide at least a portion of the plurality of vaporizing control parameters via the remote device 130. For example, a health care professional treating the user of the personal vaporizing device 100 may determine an amount of a medicinal agent to be included in the vaporizable liquid composition according to the characteristics of the user or the condition of the user. The health care profession may also determine the parameters for vaporizing the liquid composition. The health care professional would then input the data into the remote device 130, which would be then be transmitted to the input/output device 112 via communication link 132 and then to the processor 102 for processing thereof.

In accordance with present embodiments disclosed herein, the personal vaporizing device 100 has multiple methods of activation dependent upon user preference, a characteristic of a user of the device, and/or an operating characteristic of the device and/or vaporizable material. While reference is made to personal vaporizing device 100, it is to be understood that the personal vaporizer may be any personal vaporizing device in accordance with the present disclosure.

In one embodiment, the personal vaporizing device 100 may be activated in accordance with at least one device activation parameter supplied by a user thereof. In such embodiment, a user of the personal vaporizing device 100 provides at least one device activation parameter for activating the personal vaporizing device 100 and/or the vaporizing component 108. In one embodiment, a user may input desired device activation parameters via a user interface associated with the input/output device 112. The input/output device 112 may include the functionality to allow an associated user to select parameters, features or other options for the device activation parameters. In another embodiment, at least a portion of the device activation parameters may be provided via a user interface associated with the remote device 130 and then transmitted to the input/output device 112 via communication link 132.

As an example, the device activation parameters provided by a user thereof may include, but are not limited to, a type of activation mode, a location of the personal vaporizing device 100, a time of day, a type of vaporizable liquid material stored in the container 110, a characteristic of the container 110, desired vapor output (mixture, temperature, amount of vapor, etc.), a characteristic of the personal vaporizing device 100, a characteristic of the mouthpiece 114 or other component of the personal vaporizing device 100, and combinations thereof. As an example, a user may desire that the personal vaporizing device 100 is activated via a selected mode of a physical or digital controller. In another example, the user may desire that the personal vaporizing device 100 is only activated at certain locations, certain times of day, or respect to certain activities of the user. In yet another example, the user may desire that the personal vaporizing device 100 is only activated provided a selected container 110, mouthpiece 114, or other component is coupled or connected to the personal vaporizing device 100. For instance, the user may desire that the personal vaporizing device 100 is only activated when a container 110 containing a specific liquid composition, such as a medication, is coupled to the personal vaporizing device 100.

In operation, the personal vaporizing device 100 may obtain a plurality of device activation parameters from a user for controlling the activation of the personal vaporizing device 100 and/or the vaporizing component 108. The processor 102 may generate at least one device activation control signal in accordance with at least a portion of the plurality of device activation parameters. The personal vaporizing device 100 and/or the vaporizing component 108 may then be activated in accordance with the at least one device activation control signal.

In another embodiment, the personal vaporizing device 100 may be activated in accordance with at least one device activation parameter associated with a mouthpiece 114 to be used in the device 100. In such embodiment, based on the mouthpiece 114 installed into the personal vaporizing device 100, the operating parameters of the personal vaporizing device 100 and/or the vaporizing component 108 will be configured in accordance with the parameters associated with such mouthpiece 114. As an example, the device activation parameters provided by a specific mouthpiece may include, but are not limited to, a type of activation mode, a type of vaporizable liquid material stored in the container 110, a characteristic of the container 110, desired vapor output (mixture, temperature, amount of vapor, etc.), and combinations thereof.

In one embodiment, the mouthpiece 114 may supply the at least one device activation parameter based on a physical configuration of the mouthpiece 114 and/or a physical connection between the mouthpiece 114 and the personal vaporizing device 100. In one embodiment, a specific physical connection between the mouthpiece 114 and the personal vaporizing device 100 may allow certain operation modes and/or inhibit certain operation modes.

In another embodiment, the mouthpiece 114 may be operatively connected to the processor 102, such that the when the mouthpiece 114 is inserted into the personal vaporizing device 110, the at least one device activation parameter is transmitted to the processor 102 for processing thereof. In such embodiment, the device activation parameters provided by the specific mouthpiece 114 may only allow certain operations or may inhibit certain operations.

In yet another embodiment, the personal vaporizing device 100 includes a component sensing module 122 operatively connected to the processor 102. The component sensing module 122 is configured to be in contact with selected components of the personal vaporizing device 100 and to detect a plurality of physical and/or operating parameter data of such components. The component sensing module 122 transmits the detected data to the processor 102 for processing thereby. The component sensing module 122 is operable to detect at least one of audio data, optical data, thermal data, pressure data, electrical data, mechanical/physical data, biochemical/chemical data, and the like, and combinations thereof. The component sensing module 122 may comprise one or more of, a biochemical/chemical sensor, a thermal sensor, a radiation sensor, a mechanical sensor, an optical sensor, a magnetic sensor, an electrical sensor, combinations thereof and the like.

In one embodiment, the component sensing module 122 is in contact with a specific mouthpiece 114 inserted into the personal vaporizing device 100 and detects a plurality of operating parameter data associated with the mouthpiece 114 for transmission to the processor 102. The operating parameter data preferably includes at least one device activation parameter for controlling the activation of the personal vaporizing device 100 and/or the vaporizing component 108. In such embodiment, the device activation parameters provided by the specific mouthpiece 114 may only allow certain operations or may inhibit certain operations.

In another embodiment, the component sensing module 122 is in contact with a specific mouthpiece 114 inserted into the personal vaporizing device 100 and detects a plurality of physical parameter data associated with the mouthpiece 114 for transmission to the processor 102. Based on at least a portion of the received physical parameter data, the processor 102 determines operating parameter data associated with the mouthpiece 114. In a preferred embodiment, the operating parameter data includes at least one device activation parameter for controlling the activation of the personal vaporizing device 100 and/or the vaporizing component 108.

In operation, the personal vaporizing device 100 may obtain a plurality of device activation parameters from the mouthpiece 114 installed into the personal vaporizing device 100 for controlling the activation of the personal vaporizing device 100 and/or the vaporizing component 108. The processor 102 may generate at least one device activation control signal in accordance with at least a portion of the plurality of device activation parameters. The personal vaporizing device 100 and/or the vaporizing component 108 may then be activated in accordance with the at least one device activation control signal.

In another embodiment, the personal vaporizing device 100 may be activated in accordance with at least one device activation parameter associated with a container 110 to be used in the device 100. In such embodiment, based on the container 110 installed into the personal vaporizing device 100, the operating parameters of the personal vaporizing device 100 and/or the vaporizing component 108 will be configured in accordance with the parameters associated with such container 110. As an example, the device activation parameters provided by a specific container may include, but are not limited to, a type of activation mode, desired vapor output (mixture, temperature, amount of vapor, etc.), and combinations thereof.

In one embodiment, the container 110 may supply the at least one device activation parameter based on a physical configuration of the container 110 and/or a physical connection between the container 110 and the personal vaporizing device 100. In one embodiment, a specific physical connection between the container 110 and the personal vaporizing device 100 may allow certain operation modes and/or inhibit certain operation modes.

In another embodiment, the container 110 may be operatively connected to the processor 102, such that the when the container 110 is inserted into the personal vaporizing device 110, the at least one device activation parameter is transmitted to the processor 102 for processing thereof. In such embodiment, the device activation parameters provided by the specific container 110 may only allow certain operations or may inhibit certain operations.

In yet another embodiment, the component sensing module 122 is in contact with a specific container 110 inserted into the personal vaporizing device 100 and detects a plurality of operating parameter data associated with the container 110 for transmission to the processor 102. The operating parameter data preferably includes at least one device activation parameter for controlling the activation of the personal vaporizing device 100 and/or the vaporizing component 108. In such embodiment, the device activation parameters provided by the specific container 110 may only allow certain operations or may inhibit certain operations.

In another embodiment, the component sensing module 122 is in contact with a specific container 110 inserted into the personal vaporizing device 100 and detects a plurality of physical parameter data associated with the container 110 for transmission to the processor 102. Based on at least a portion of the received physical parameter data, the processor 102 determines operating parameter data associated with the container 110. In a preferred embodiment, the operating parameter data includes at least one device activation parameter for controlling the activation of the personal vaporizing device 100 and/or the vaporizing component 108.

In operation, the personal vaporizing device 100 may obtain a plurality of device activation parameters from the container 110 installed into the personal vaporizing device 100 for controlling the activation of the personal vaporizing device 100 and/or the vaporizing component 108. The processor 102 may generate at least one device activation control signal in accordance with at least a portion of the plurality of device activation parameters. The personal vaporizing device 100 and/or the vaporizing component 108 may then be activated in accordance with the at least one device activation control signal.

While reference is made to obtaining activation parameters from a specific mouthpiece 114 or a specific container 110 installed into the personal vaporizing device 100, it is to be understood that a combination of a specific mouthpiece 114 together with a specific container 110 may provide at least one device activation parameter for controlling activation of the personal vaporizing device 100. For example, a specific mouthpiece 114 in combination with a first type of container 110 may have one set of device activation parameters, and such mouthpiece 114 in combination with a second type of container 110 may have a second set of device activation parameters.

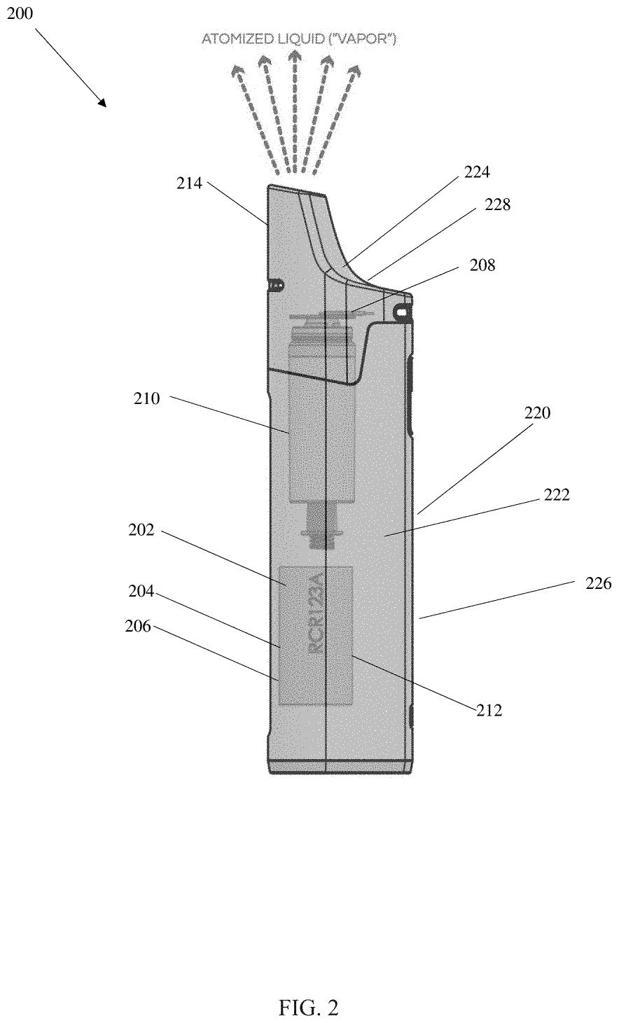

FIG. 2 illustrates one embodiment of a personal vaporizer 200. The vaporizer 200 may be, for example, an e-cigarette, an e-cigar, an electronic vapor device, a modified vapor device “mod,” a micro-sized personal vaporizing device, and the like. The vaporizer 200 includes a cylindrically-shaped housing 220 having a mouthpiece 214 and an elongated region 224 opposite the mouthpiece 214. The personal vaporizer 200 comprises a processor 202 for controlling the operation of the personal vaporizer 200. The personal vaporizer 200 may also include a memory device 204 coupled to the processor 202, a network access device 206 allowing the personal vaporizer 200 to be connected to one or more ancillary devices, and an input/output device 212 for exchanging data with a user or other device.

The personal vaporizer 200 further comprises a vaporizing component 208 operatively connected to the processor 202 for controlling the operation of the vaporizing component 208. The vaporizing component 208 receives vaporizable liquid composition from container 210 contained within the housing 220 and vaporizes at least a portion of the liquid composition to generate a vapor therefrom.

In one embodiment, the personal vaporizer 200 includes a component sensing module 222 operatively connected to the processor 202. The component sensing module 222 is configured to be in contact with selected components of the personal vaporizer 200 and to detect a plurality of physical and/or operating parameter data of such components. In a preferred embodiment, the component sensing module 222 is in contact with at least one of a mouthpiece 214 and/or a container 210 installed into the personal vaporizer 200.

In one embodiment, the component sensing module 222 may detect operating parameter data associated with the mouthpiece 214 and/or the container 210 for transmission to the processor 202. In another embodiment, the component sensing module 222 may detect physical parameter data associated with the mouthpiece 214 and/or container 210 for transmission to the processor 202. Based on at least a portion of the received physical parameter data, the processor 202 determines operating parameter data associated with the mouthpiece 214 and/or container 210. In a preferred embodiment, the operating parameter data includes at least one device activation parameter for controlling the activation of the personal vaporizer 200 and/or the vaporizing component 208. The processor 202 may generate at least one device activation control signal in accordance with at least a portion of the plurality of device activation parameters. The personal vaporizer 200 and/or the vaporizing component 208 may then be activated in accordance with the at least one device activation control signal.

The personal vaporizer 200 further includes a display 226 on the cylindrically-shaped housing 220 and operatively connected to the processor 202. The display 226 may be configured to display information associated with the operation of the personal vaporizer 200. The display may be a single LED or may be more complicated, such as but not limited to: a multi-colored LED light (wherein different colors mean different things), a bank or array of LED lights, a 2D LED display, and the like, and combinations thereof. The display 226 may also prompt a user for actions required to operate the personal vaporizer 200.

The personal vaporizer 200 may also comprise a push button control 228 disposed on the elongated region 224 of the cylindrically-shaped housing 220, and operatively connected to the processor 202 and/or the input/output device 212. The push button control 228 provides a user the ability to control various operations of the personal vaporizer 200 by activating the push button control 228. As an example, such operations may include, but are not limited to, powering the personal vaporizer 200 on and off, activating/deactivating the vaporizing component 208, selecting a mode of operation for the vaporizing component 208, activating a security function of the personal vaporizer 208, and the like, and combinations thereof.

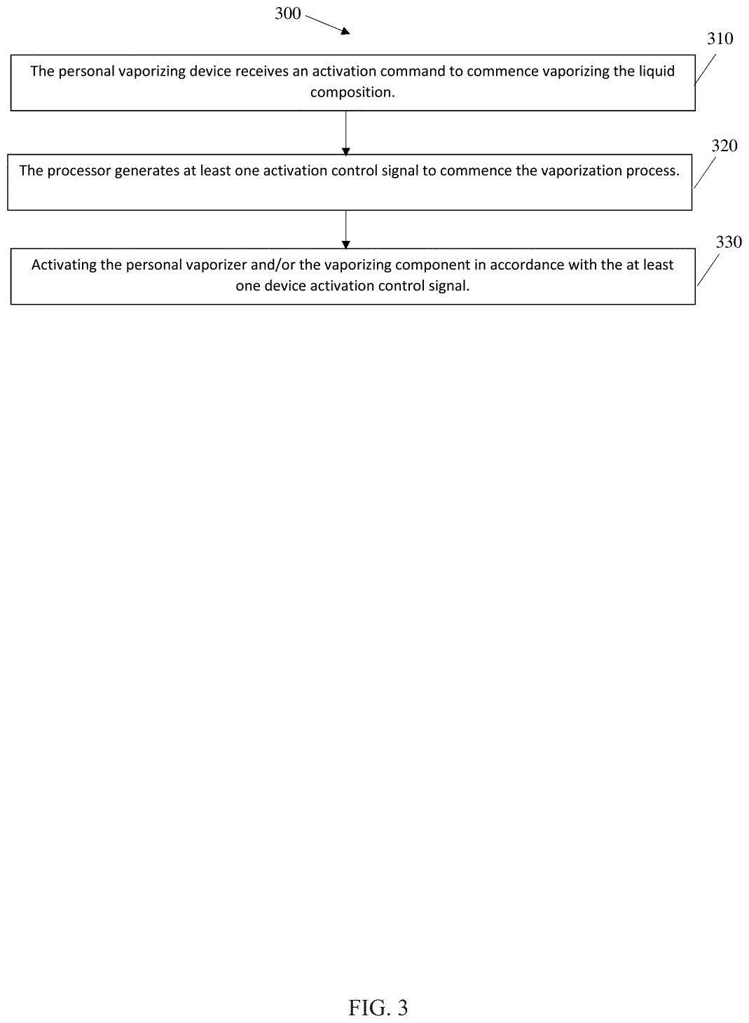

In an embodiment, illustrated in FIG. 3, a method 300 may be provided for activating a personal vaporizing device, such as personal vaporizer 200. The method comprises step 310 of receiving at least one device activation parameter from a user of the personal vaporizer 200. The device activation parameter may be obtained by any suitable means. In one embodiment, a user may input desired device activation parameters via a user interface associated with the input/output device 212. The input/output device 112 may include the functionality to allow an associated user to select parameters, features or other options for the device activation parameters. In another embodiment, at least a portion of the device activation parameters may be provided from a remote device via network access device 206. As an example, the device activation parameters provided by a user thereof may include, but are not limited to, a type of activation mode, a location of the personal vaporizer, a time of day, a type of vaporizable liquid material stored in the container, a characteristic of the container, desired vapor output (mixture, temperature, amount of vapor, etc.), a characteristic of the personal vaporizer, a characteristic of a component of the personal vaporizer, and combinations thereof.

The method further comprises step 320 of generating, by the processor 202, at least one device activation control signal in accordance with at least a portion of the plurality of device activation parameters.

The method also comprises step 330 of activating the personal vaporizer 200 and/or the vaporizing component 208 in accordance with the at least one device activation control signal. In one embodiment, the personal vaporizer 200 may be activated by activating the push button control 228. In another embodiment, the personal vaporizer 200 may be activated by receiving an activation confirmation signal from a remote device via network access link 206. In yet another embodiment, the personal vaporizer 200 may be activated by receiving an activation confirmation signal from the component sensing module 222. While reference is made to activation by any single component, it is to be understood the personal vaporizer 200 may be activated by any combination of components.

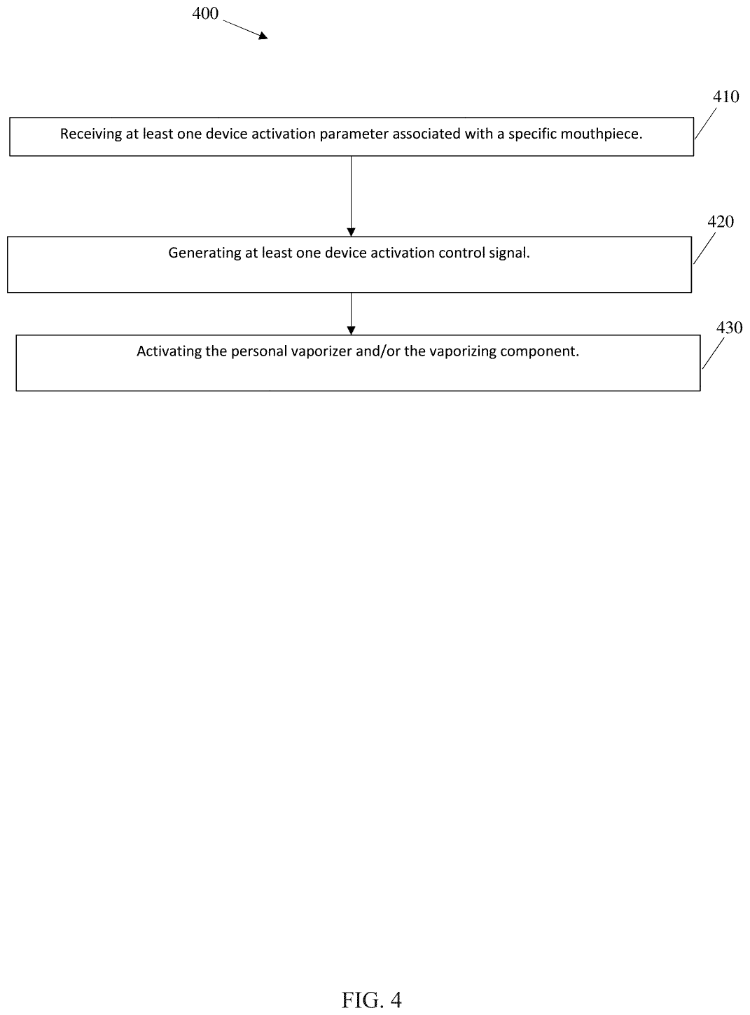

In an embodiment, illustrated in FIG. 4, a method 400 may be provided for activating a personal vaporizing device, such as personal vaporizer 200. The method comprises step 410 of receiving at least one device activation parameter associated with a specific mouthpiece 214 installed in personal vaporizer 200. As an example, the device activation parameters provided by a specific mouthpiece may include, but are not limited to, a type of activation mode, a type of vaporizable liquid material stored in the container, a characteristic of the container, desired vapor output (mixture, temperature, amount of vapor, etc.), and combinations thereof.

In one embodiment, the mouthpiece 214 may supply the at least one device activation parameter based on a physical configuration of the mouthpiece 214 and/or a physical connection between the mouthpiece 214 and the personal vaporizer 200. In another embodiment, the mouthpiece 214 may be operatively connected to the processor 202, such that the when the mouthpiece 214 is inserted into the personal vaporizer 210, the at least one device activation parameter is transmitted to the processor 202 for processing thereof.

In one embodiment, the component sensing module 222 is in contact with a specific mouthpiece 214 inserted into the personal vaporizer 200 and detects a plurality of operating parameter data associated with the mouthpiece 214 for transmission to the processor 202. In another embodiment, the component sensing module 222 is in contact with a specific mouthpiece 214 inserted into the personal vaporizer 200 and detects a plurality of physical parameter data associated with the mouthpiece 214 for transmission to the processor 202. Based on at least a portion of the received physical parameter data, the processor 202 determines operating parameter data associated with the mouthpiece 214. In a preferred embodiment, the operating parameter data includes at least one device activation parameter for controlling the activation of the personal vaporizer 200 and/or the vaporizing component 208.

The method further comprises step 420 of generating, by the processor 202, at least one device activation control signal in accordance with at least a portion of the plurality of device activation parameters.

The method also comprises step 430 of activating the personal vaporizer 200 and/or the vaporizing component 208 in accordance with the at least one device activation control signal. In one embodiment, the personal vaporizer 200 may be activated by activating the push button control 228. In another embodiment, the personal vaporizer 200 may be activated by receiving an activation confirmation signal from a remote device via network access link 206. In yet another embodiment, the personal vaporizer 200 may be activated by receiving an activation confirmation signal from the component sensing module 222. While reference is made to activation by any single component, it is to be understood the personal vaporizer 200 may be activated by any combination of components.

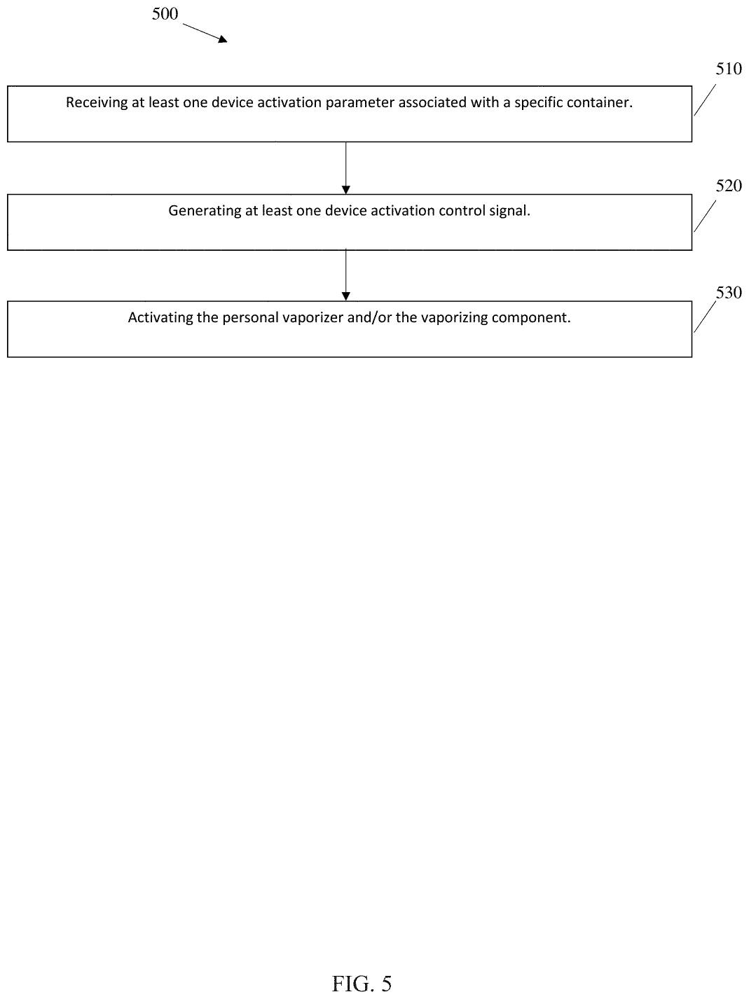

In an embodiment, illustrated in FIG. 5, a method 500 may be provided for activating a personal vaporizing device, such as personal vaporizer 200. The method comprises step 510 of receiving at least one device activation parameter associated with a specific container 210 installed in personal vaporizer 200. As an example, the device activation parameters provided by a specific mouthpiece may include, but are not limited to, a type of activation mode, desired vapor output (mixture, temperature, amount of vapor, etc.), and combinations thereof.

In one embodiment, the container 210 may supply the at least one device activation parameter based on a physical configuration of the container 210 and/or a physical connection between the container 210 and the personal vaporizer 200. In another embodiment, the container 210 may be operatively connected to the processor 202, such that the when the container 210 is inserted into the personal vaporizer 210, the at least one device activation parameter is transmitted to the processor 202 for processing thereof.

In one embodiment, the component sensing module 222 is in contact with a specific container 210 inserted into the personal vaporizer 200 and detects a plurality of operating parameter data associated with the container 210 for transmission to the processor 202. In another embodiment, the component sensing module 222 is in contact with a specific container 210 inserted into the personal vaporizer 200 and detects a plurality of physical parameter data associated with the container 210 for transmission to the processor 202. Based on at least a portion of the received physical parameter data, the processor 202 determines operating parameter data associated with the container 210. In a preferred embodiment, the operating parameter data includes at least one device activation parameter for controlling the activation of the personal vaporizer 200 and/or the vaporizing component 208.

The method further comprises step 520 of generating, by the processor 202, at least one device activation control signal in accordance with at least a portion of the plurality of device activation parameters.

The method also comprises step 530 of activating the personal vaporizer 200 and/or the vaporizing component 208 in accordance with the at least one device activation control signal. In one embodiment, the personal vaporizer 200 may be activated by activating the push button control 228. In another embodiment, the personal vaporizer 200 may be activated by receiving an activation confirmation signal from a remote device via network access link 206. In yet another embodiment, the personal vaporizer 200 may be activated by receiving an activation confirmation signal from the component sensing module 222. While reference is made to activation by any single component, it is to be understood the personal vaporizer 200 may be activated by any combination of components.

While reference is made to obtaining activation parameters from a specific mouthpiece 214 or a specific container 210 installed into the personal vaporizer 200, it is to be understood that a combination of a specific mouthpiece 214 together with a specific container 210 may provide at least one device activation parameter for controlling activation of the personal vaporizer 200. As such, the methods described herein may include obtaining activation parameters for a combination of a specific mouthpiece 214 combined with a specific container 210.

In view of the exemplary systems described herein, methodologies that may be implemented in accordance with the disclosed subject matter have been described with reference to several flow diagrams. While for purposes of simplicity of explanation, the methodologies are shown and described as a series of blocks, it is to be understood and appreciated that the claimed subject matter is not limited by the order of the blocks, as some blocks may occur in different orders and/or concurrently with other blocks from what is depicted and described herein. Moreover, not all illustrated blocks may be required to implement the methodologies described herein. Additionally, it should be further appreciated that the methodologies disclosed herein are capable of being stored on an article of manufacture to facilitate transporting and transferring such methodologies to computers.

Those of ordinary skill in the relevant art would further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

As used in this application, the terms “component,” “module,” “system,” and the like are intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on a server and the server may be a component. One or more components may reside within a process and/or thread of execution and a component may be localized on one computer and/or distributed between two or more computers.

As used herein, a “vapor” includes mixtures of a carrier gas or gaseous mixture (for example, air) with any one or more of a dissolved gas, suspended solid particles, or suspended liquid droplets, wherein a substantial fraction of the particles or droplets if present are characterized by an average diameter of not greater than three microns. As used herein, an “aerosol” has the same meaning as “vapor,” except for requiring the presence of at least one of particles or droplets. A substantial fraction means 10% or greater; however, it should be appreciated that higher fractions of small (<3 micron) particles or droplets may be desirable, up to and including 100%. It should further be appreciated that, to simulate smoke, average particle or droplet size may be less than three microns, for example, may be less than one micron with particles or droplets distributed in the range of 0.01 to 1 micron. A vaporizer may include any device or assembly that produces a vapor or aerosol from a carrier gas or gaseous mixture and at least one vaporizable material. An aerosolizer is a species of vaporizer, and as such is included in the meaning of vaporizer as used herein, except where specifically disclaimed.

Various embodiments presented in terms of systems may comprise a number of components, modules, and the like. It is to be understood and appreciated that the various systems may include additional components, modules, etc. and/or may not include all of the components, modules, etc. discussed in connection with the figures. A combination of these approaches may also be used.

In addition, the various illustrative logical blocks, modules, and circuits described in connection with certain embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, system-on-a-chip, or state machine. A processor may also be implemented as a combination of computing devices, e.g., a combination of a DSP and a microprocessor, a plurality of microprocessors, one or more microprocessors in conjunction with a DSP core, or any other such configuration.

Operational embodiments disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD- ROM, a DVD disk, or any other form of storage medium known in the art. An exemplary storage medium is coupled to the processor such the processor may read information from, and write information to, the storage medium. In the alternative, the storage medium may be integral to the processor. The processor and the storage medium may reside in an ASIC or may reside as discrete components in another device.

Furthermore, the one or more versions may be implemented as a method, apparatus, or article of manufacture using standard programming and/or engineering techniques to produce software, firmware, hardware, or any combination thereof to control a computer to implement the disclosed embodiments. Non-transitory computer readable media may include but are not limited to magnetic storage devices (e.g., hard disk, floppy disk, magnetic strips), optical disks (e.g., compact disk (CD), digital versatile disk (DVD)), smart cards, and flash memory devices (e.g., card, stick). Those skilled in the art will recognize many modifications may be made to this configuration without departing from the scope of the disclosed embodiments.

The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those of ordinary skill in the art that various modifications and variations may be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A personal vaporizer comprising:
a device processor operable to control the personal vaporizer, wherein the device processor is operable to generate an activation command to initiate a vaporization process;
a container configured to store a vaporizable liquid composition;
an ultrasonic vaporizing component operatively coupled to the device processor and controlled in part by the device processor, wherein the ultrasonic vaporizing component is in fluid communication with the container for receiving a selected amount of vaporizable liquid composition from the container, wherein the ultrasonic vaporizing component comprises an ultrasonic vibration element operable to produce ultrasonic vibrations to vaporize at least a portion of the vaporizable liquid composition received therein;
a mouthpiece coupled to the ultrasonic vaporizing component and configured to receive vapor generated by the ultrasonic vaporizing component, the mouthpiece operable to expel the generated vapor from the ultrasonic vaporizing component;
an input/output device operatively coupled to the device processor; wherein the input/output device is configured to receive a plurality of data for transmission to the device processor, wherein the input/output device is configured to transmit a plurality of data generated by the device processor; and
a power source operatively coupled to the ultrasonic vaporizing component, wherein the power source is operable to generate an electric current for operation of the ultrasonic vaporizing component;
wherein the device processor is operable to:
receive a plurality of device activation parameters for controlling activation of the vaporization process;
generate at least one device activation control signal in accordance with at least a portion of the plurality of device activation parameters; and
transmit the at least one device activation control signal to the ultrasonic vaporizing component to initiate the vaporization process in accordance the at least one device activation control signal.

2. The personal vaporizer of claim 1, wherein the input/output device comprises a user interface, wherein the device processor is operable to receive at least a portion of the plurality of device activation parameters from an associated user via the user interface.

3. The personal vaporizer of claim 1, wherein the input/output device is configured to receive at least a portion of the plurality of device activation parameters for controlling activation of a vaporization process from a remote device.

4. The personal vaporizer of claim 1, wherein the plurality of device activation parameters is selected from the group consisting of type of activation mode, a location of the personal vaporizer, a time of day, a type of vaporizable liquid composition stored in the container, an operational parameter of the container, desired vapor output, an operational parameter of the mouthpiece, and combinations thereof.

5. The personal vaporizer of claim 1, wherein the device processor is further operable to:
detect a plurality of status data associated with at least one operational characteristic of at least one of the container, the mouthpiece, the ultrasonic vaporizing component and combinations thereof;
determine, based on the at least a portion of the detected status data, at least one operational parameter of the personal vaporizer;
determine, based on the at least one operational parameter, at least one device activation parameter for controlling activation of the vaporization process; and
generate at least one device activation control signal in accordance with the at least one device activation parameter.

6. The personal vaporizer of claim 1, further comprising a sensing component operatively connected to the device processor and controlled in part by the device processor, wherein the sensing component is operable to detect a plurality of status data associated with at least one operational characteristic of the personal vaporizer and transmit at least a portion of the detected status data to the device processor.

7. The personal vaporizer of claim 6, wherein the device processor is further operable to:
receive at least a portion of the detected status data;
determine, based on the received status data, at least one operational parameter of the personal vaporizer;
determine, based on the at least one operational parameter, at least one device activation parameter for controlling activation of the vaporization process; and
generate at least one device activation control signal in accordance with the at least one device activation parameter.

8. The personal vaporizer of claim 6,
wherein the sensing component is operable to detect a plurality of status data associated at least one operational characteristic of the mouthpiece and transmit at least a portion of the detected mouthpiece status data to the device processor;
wherein the device processor is further operable to:
receive at least a portion of the detected mouthpiece status data;
determine, based on the received mouthpiece status data, at least one operational parameter of the mouthpiece;
determine, based on the at least one operational parameter, at least one device activation parameter for controlling activation of the vaporization process; and
generate at least one device activation control signal in accordance with the at least one device activation parameter.

9. The personal vaporizer of claim 6,
wherein the sensing component is operable to detect a plurality of status data associated at least one operational characteristic of the container and transmit at least a portion of the detected container status data to the device processor;
wherein the device processor is further operable to:
receive at least a portion of the detected container status data;
determine, based on the received container status data, at least one operational parameter of the container;
determine, based on the at least one operational parameter, at least one device activation parameter for controlling activation of the vaporization process; and
generate at least one device activation control signal in accordance with the at least one device activation parameter.

10. The personal vaporizer of claim 6, wherein the sensing component is selected from the group of sensing components consisting of: a biochemical/chemical sensor, a thermal sensor, a radiation sensor, a mechanical sensor, an optical sensor, a magnetic sensor, an electrical sensor, and combinations thereof.

11. A method for activating a personal vaporizer to initiate a vaporization process, the personal vaporizer comprising,
(a) a device processor operable for controlling the personal vaporizer;
(b) a container configured to store a vaporizable liquid composition;
(c) an ultrasonic vaporizing component comprising an ultrasonic vibration element operable to produce ultrasonic vibrations to vaporize at least a portion of the vaporizable liquid composition received therein;
(d) a mouthpiece configured to receive vapor generated by the ultrasonic vaporizing component and expel the generated vapor from the ultrasonic vaporizing component;
(e) an input/output device configured to receive a plurality of data for transmission to the device processor and to transmit a plurality of data generated by the device processor; and
(f) a power source operable to generate a variable strength electrical current for operation of the ultrasonic vaporizing component, the method comprising:
receiving, at the device processor a plurality of device activation parameters for controlling activation of the vaporization process;
generating, via the device processor at least one device activation control signal in accordance with at least a portion of the plurality of device activation parameters;
transmitting, via the input/output device, the at least one device activation control signal to the ultrasonic vaporizing component to initiate the vaporization process in accordance the at least one device activation control signal;
initiating, by the ultrasonic vaporizing component, a vaporization process to vaporize at least a portion of the vaporizable liquid composition.

12. The method of claim 11, wherein the input/output device comprises a user interface, the method further comprising receiving at least a portion of the plurality of device activation parameters from an associated user via the user interface.

13. The method of claim 11, further comprising receiving, via the input/output device, at least a portion of the plurality of device activation parameters for controlling activation of a vaporization process from a remote device.

14. The method of claim 11, wherein the plurality of device activation parameters is selected from the group consisting of type of activation mode, a location of the personal vaporizer, a time of day, a type of vaporizable liquid composition stored in the container, an operational parameter of the container, desired vapor output, an operational parameter of the mouthpiece, and combinations thereof.

15. The method of claim 11, further comprising:
detecting, via the device processor, a plurality of status data associated with at least one operational characteristic of at least one of the container, the mouthpiece, the ultrasonic vaporizing component and combinations thereof;
determining, via the device processor, based on the at least a portion of the detected status data, at least one operational parameter of the personal vaporizer;
determining, via the device processor, based on the at least one operational parameter, at least one device activation parameter for controlling activation of the vaporization process; and
generating, via the device processor, at least one device activation control signal in accordance with the at least one device activation parameter.

16. The method of claim 15, wherein the personal vaporizer further comprises a sensing component, the method comprising detecting, via the sensing component, a plurality of status data associated with at least one operational characteristic of the personal vaporizer and transmitting at least a portion of the detected status data to the device processor.

17. The method of claim 16, further comprising:
receiving, at the device processor, at least a portion of the detected status data;
determining, via the device processor, based on the received status data, at least one operational parameter of the personal vaporizer;
determining, via the device processor, based on the at least one operational parameter, at least one device activation parameter for controlling activation of the vaporization process; and
generating, via the device processor, at least one device activation control signal in accordance with the at least one device activation parameter.

18. The method of claim 16, further comprising
detecting, by the sensing component, a plurality of status data associated at least one operational characteristic of the mouthpiece and transmitting at least a portion of the detected mouthpiece status data to the device processor;
determining, by the device processor, based on the received mouthpiece status data, at least one operational parameter of the mouthpiece;
determining, by the device processor, based on the at least one operational parameter, at least one device activation parameter for controlling activation of the vaporization process; and
generating, by the device processor, at least one device activation control signal in accordance with the at least one device activation parameter.

19. The method of claim 16, further comprising
detecting, by the sensing component, a plurality of status data associated at least one operational characteristic of the container and transmitting at least a portion of the detected container status data to the device processor;
determining, by the device processor, based on the received container status data, at least one operational parameter of the container;
determining, by the device processor, based on the at least one operational parameter, at least one device activation parameter for controlling activation of the vaporization process; and
generating, by the device processor, at least one device activation control signal in accordance with the at least one device activation parameter.

20. A personal vaporizer comprising:
a device processor operable to control the personal vaporizer, wherein the device processor is operable to generate an activation command to initiate a vaporization process;
a container configured to store a vaporizable liquid composition;
an ultrasonic vaporizing component operatively coupled to the device processor and controlled in part by the device processor, wherein the ultrasonic vaporizing component is in fluid communication with the container for receiving a selected amount of vaporizable liquid composition from the container, wherein the ultrasonic vaporizing component comprises an ultrasonic vibration element operable to produce ultrasonic vibrations to vaporize at least a portion of the vaporizable liquid composition received therein;
a mouthpiece coupled to the ultrasonic vaporizing component and configured to receive vapor generated by the ultrasonic vaporizing component, the mouthpiece operable to expel the generated vapor from the ultrasonic vaporizing component;
a sensing component operatively connected to the device processor and controlled in part by the device processor, wherein the sensing component is operable to detect a plurality of status data associated with at least one operational characteristic of at least one of the mouthpiece, the container, and the ultrasonic vaporizing component and combinations thereof, and transmit at least a portion of the detected status data to the device processor; and
a power source operatively coupled to the ultrasonic vaporizing component, wherein the power source is operable to generate an electric current for operation of the ultrasonic vaporizing component;
wherein the device processor is operable to:
determine, based on the received status data, at least one operational parameter of the personal vaporizer;
determine, based on the at least one operational parameter, at least one device activation parameter for controlling activation of the vaporization process;
generate at least one device activation control signal in accordance with the at least one device activation parameter; and
transmit the at least one device activation control signal to the ultrasonic vaporizing component to initiate the vaporization process in accordance the at least one device activation control signal.
